(19) United States
(12) Patent Application Publication
(10) Pub. No.: US 2024/0188894 A1
(43) Pub. Date: Jun. 13, 2024

(54) Visualizing Performance of Catheter Electrodes

(71) Applicant: Biosense Webster (Israel) Ltd., Yokneam (IL)

(72) Inventors: Yair PALTI, Herzelia (IL); Vadim GLINER, Haifa (IL); Assaf GOVARI, Haifa (IL); Israel ZILBERMAN, Yokneam (IL)

(21) Appl. No.: 18/444,229

(22) Filed: Feb. 16, 2024

Related U.S. Application Data
(63) Continuation of application No. 17/129,687, filed on Dec. 21, 2020, now Pat. No. 11,918,383.

(51) Int. Cl. A61B 5/00; A61B 5/287; A61B 5/339; A61B 5/346; A61B 5/367; G16H 20/40; G16H 40/63

(52) U.S. Cl. CPC A61B 5/7221; A61B 5/287; A61B 5/339; A61B 5/346; A61B 5/367; A61B 5/6858; A61B 5/6885; A61B 5/7264; G16H 20/40; G16H 40/63; A61B 5/7267; A61B 5/7435; A61B 2562/0209

(57) ABSTRACT

A system for electrophysiological measurement includes a probe having a distal end configured for insertion into a body cavity of a living subject and including an array of electrodes that are disposed along the distal end and are configured to contact tissue at multiple locations within the body cavity. A processor is configured to acquire signals from the electrodes over a period of time during which the probe moves within the body cavity, to compute, in response the signals, metrics that are indicative of a respective quality of contact between each of the electrodes and the tissue over the period of time, and to output an indication of the metrics to a user of the system.

### CROSS-REFERENCE TO RELATED APPLICATIONS

This application is a continuation of prior filed U.S. patent application Ser. No. 17/129,687 filed on Dec. 21, 2020 (Attorney Docket No. 253757.000309 (BIO6453USNP1)), which prior application is hereby incorporated by reference as if set forth in full into this application.

### FIELD OF THE INVENTION

The present invention relates generally to apparatus and methods for sensing and mapping of electrophysiological (EP) signals, and particularly to methods for evaluating the operation of such apparatus.

### BACKGROUND

In cardiac electroanatomical mapping systems that are known in the art, an operator—typically a physician—inserts a catheter through a patient's vascular system into a chamber of the heart. An electrode or electrode assembly at the distal end of the catheter contacts the myocardial tissue in the chamber and receives electrical signals from the tissue, which are conveyed through the catheter to a mapping console. The operator manipulates the catheter within the heart in order to acquire signals from many points within the heart chamber, thus enabling the console to construct a map showing the physical structure of the walls of the heart chamber and the distribution of electrical activity over the walls.

As the operator cannot see the distal end of the catheter in the heart chamber, a number of techniques have been developed to assist the operator in visualizing and understanding the process of EP signal acquisition. For example, U.S. Pat. No. 10,617,317 describes a method for highlighting an electrode image according to an electrode signal. A graphical image of a heart of a patient is presented on a display screen, including icons representing a catheter that is positioned within the heart and an electrode on the catheter, while the electrode is in contact with tissue at a location in the heart. The method further includes acquiring, using the electrode, electrical signals from the tissue at the location, and processing the acquired signals so as to detect an occurrence of a predefined signal feature in the acquired signals. The method also includes, upon detecting the occurrence of the predefined signal feature, modifying a visual feature of at least one of the icon representing the electrode and the icon representing the catheter on the display screen.

As another example, U.S. Pat. No. 10,582,872 describes a method and system for visualization of electrophysiology information sensed by electrodes on a catheter. The method includes recording times of electrode signal acquisition, designating a reference electrode signal acquisition, assigning a relative time to each recorded time of electrode signal acquisition relative to the reference electrode signal acquisition, identifying the electrodes with signal acquisition, correlating assigned relative times to identified electrodes to generate a sequence of electrode signal acquisitions, and generating a visual representation of the sequence of electrode signal acquisitions generating a visual representation with a graphical image of the electrodes, wherein individual electrodes are visually marked to represent the sequence of electrode signal acquisitions.

### SUMMARY

Embodiments of the present invention that are described hereinbelow provide improved methods and systems for visualization of EP signal acquisition.

There is therefore provided, in accordance with an embodiment of the invention, a system for electrophysiological measurement, including a probe having a distal end configured for insertion into a body cavity of a living subject and including an array of electrodes that are disposed along the distal end and are configured to contact tissue at multiple locations within the body cavity. A processor is configured to acquire signals from the electrodes over a period of time during which the probe moves within the body cavity, to compute, in response the signals, metrics that are indicative of a respective quality of contact between each of the electrodes and the tissue over the period of time, and to output an indication of the metrics to a user of the system.

In a disclosed embodiment, the probe includes a catheter, and the distal end is configured for insertion into a chamber of a heart of the living subject.

Additionally or alternatively, the distal end of the probe includes a flexible structure on which the electrodes are arrayed, and the metrics are indicative of a contact between different parts of the flexible structure and the tissue. In some embodiments, the structure includes multiple flexible spines along which the electrodes are disposed.

In some embodiments, the processor is configured to render to a display a graphical icon representing the distal end and to incorporate in the graphical icon visual indications of the metrics at respective locations of the electrodes on the distal end. In a disclosed embodiment, the metrics are represented by color-coding of the respective locations of the electrodes on the graphical icon.

In one embodiment, the metrics are indicative of a number of valid signals acquired by each of the electrodes from the tissue over the period of time. Typically, the processor is configured to apply one or more filtering criteria to the signals in order to classify as valid a respective first set of the signals acquired from each of the electrodes while classifying as invalid a respective second set of the signals acquired by each of the electrodes.

In other embodiments, the metrics are indicative of a respective duration during which each of the electrodes was in contact with the tissue in the body cavity over the period of time. In one such embodiment, the signals are indicative of an electrophysiological activity within the tissue, and the processor is configured to distinguish between local signals acquired by the electrodes that are in contact with the tissue and far-field signals acquired by the electrodes that are not in contact with the tissue, and to find the duration during which each of the electrodes is in contact with the tissue in response a relation between the local and far-field signals acquired by each of the electrodes over the period of time.

There is also provided, in accordance with an embodiment of the invention, a method for electrophysiological measurement, which includes inserting into a body cavity of a living subject a probe having a distal end including an array of electrodes that are disposed along the distal end and are configured to contact tissue at multiple locations within the body cavity. Signals are acquired from the electrodes within the body cavity over a period of time during which the probe moves within the body cavity. Responsively to the signals, metrics are computed, wherein the metrics are indicative of a respective quality of contact between each of the electrodes and the tissue over the period of time. An indication of the metrics is outputted to a user of the system.

The present invention will be more fully understood from the following detailed description of the embodiments thereof, taken together with the drawings in which:

### BRIEF DESCRIPTION OF THE DRAWINGS

FIG. 1 is a schematic pictorial illustration of a system for electroanatomical mapping, in accordance with an embodiment of the present invention;

FIG. 2 is a schematic illustration of a graphical icon showing performance of electrodes on a catheter used in acquiring EP signals, in accordance with an embodiment of the present invention; and

FIG. 3 is a flow chart that schematically illustrates a method for assessing and visualizing electrode performance, in accordance with an embodiment of the present invention.

### DETAILED DESCRIPTION OF EMBODIMENTS

### Overview

To produce an accurate electroanatomical map of a heart chamber, a mapping system typically acquires electrical signals from hundreds or even thousands of different points along the wall of the chamber. To reduce the time needed to acquire this large volume of data, mapping systems commonly use catheters having many electrodes at their distal ends, which are capable of sensing respective signals simultaneously at different, respective locations within the heart chamber. The electrodes are typically arrayed along a flexible structure at the distal end of the catheter, such as a balloon or a structure with multiple flexible spines along which the electrodes are disposed, such as a basket or a multi-arm assembly.

In typical operation, not all of the electrodes will be in contact with the tissue at any given time. The signal received by a catheter electrode that is not in contact with tissue in the heart is generally dominated by the far-field signal transmitted through the blood pool in which the electrode is immersed. This far-field component is of limited diagnostic value. When the catheter electrode is in contact with the heart tissue, the amplitude of the signal derives mainly from local tissue conductivity, while the far-field contribution is minor.

Thus, for efficient, accurate EP measurement and mapping, it is generally desirable that as many electrodes as possible make contact with the tissue at all times during the procedure, and that the contact be of good quality so that the signals are suitable for incorporation in the map. By the same token, when a new catheter is in development, it is important to the designer to understand how well each of the electrodes is performing in terms of consistency of tissue contact in order to optimize the distal structure of the catheter and the placement of the electrodes on this structure. Although it is possible to evaluate the performance of any single electrode by observing the signals that it collects, the volume of data provided by the signals from the entire array of electrodes at any given time is far too large for the operator or designer to digest. It is thus difficult, for example, for the designer to assess which of the electrodes make consistently good contact with the tissue as the catheter moves through the heart chamber and which do not, in order to improve the design to achieve better, more consistent contact. There is a need for automated tools that can provide this sort of assessment and assist developers and users of catheters in improving their design and operating technique.

Embodiments of the present invention that are described here address this problem by providing a visual indication of the performance of each of the electrodes in the electrode array at the distal end of a probe, such as a catheter, in a system for electrophysiological measurement. To generate this indication, a processor acquires signals from the electrodes as the probe moves within a body cavity, such as a heart chamber. Based on the acquired signals, the processor continually evaluates the quality of contact between each electrode and the tissue in the cavity wall. For this purpose, for instance, the processor may process the signals that it acquires from each electrode in order to distinguish between local signals and far-field signals or to measure impedance though the electrodes.

For each electrode, the processor then computes a metric indicative of the quality of contact between the electrode and the tissue over a period of time during which the probe moved within the body cavity. The metric may indicate, for example, the number of valid signals acquired by each of the electrodes from the tissue over the period of time. For this purpose, for example, the processor may apply filtering criteria to the signals in order to classify the signals that meet the criteria as valid, while classifying as invalid signals that do not meet the criteria and should therefore be discarded. Methods and criteria for performing this sort of filtering are described, for example, in U.S. patent application Ser. No. 16/995,036, filed Aug. 17, 2020, which is assigned to the assignee of the present patent application and is incorporated herein by reference with a copy attached to the Appendix. Additionally or alternatively, the metric may indicate the duration during which each of the electrodes was found to be in contact with the tissue over the period of time.

The processor outputs an indication of the metrics to a user of the system. In the embodiments described below, the indication takes the form of a graphical icon representing the distal end of the probe, which the processor renders to a display. The icon includes visual indications of the metrics at the locations of the electrodes on the distal end of the probe, for example by color-coding the electrode locations on the display. This icon and/or other output enables an operator or designer to visualize the effectiveness of each of the electrodes in contacting the tissue and thus to improve either the probe design or the operating technique, as the case may be, in order to optimize the efficiency of data collection and mapping. For example, the designer may eliminate electrodes that had poor quality of contact and/or may concentrate the electrodes in areas of the probe that had good quality of contact in order to maximize the collection of valid signals relative to the available area and to the number of signal wires in the probe.

For the sake of concreteness and clarity, the embodiments that are shown in the figures and described below relate, by way of example, to a particular type of system for electroanatomical mapping and a basket catheter that can be used in such a system. The principles of the present invention, however, are by no means limited to this particular sort of catheter or system, and may similarly be applied, mutatis mutandis, to cardiac catheters of other types for diagnostic and therapeutic applications, as well as to probes used for diagnostic measurements and treatment in other body cavities. All such alternative implementations are considered to be within the scope of the present invention.

### System Description

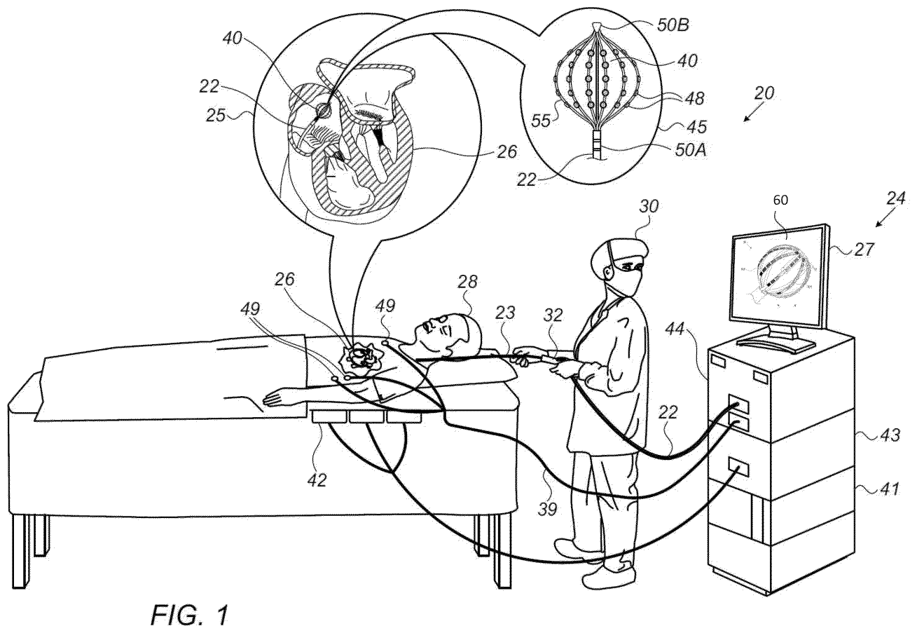

FIG. 1 is a schematic pictorial illustration of a system 20 for mapping an EP parameter in a heart 26 of a patient 28, in accordance with an embodiment of the present invention. The embodiment shown in the current figure and subsequent figures refers to an example of acquiring EP signals from a chamber of heart 26. In alternative embodiments, the values of EP parameters may be acquired using other sorts of mapping apparatus, not only from within the heart, but also from other organs and tissue, as will be apparent to those skilled in the art after reading the present description.

An operator 30 navigates a catheter 22 to a target location in heart 26 of patient 28, by manipulating a shaft 23 of the catheter, using a manipulator 32 near the proximal end of the catheter. In the pictured example, catheter 22 comprises a basket assembly 40 at its distal end, as shown in an inset 45. As seen in an inset 25, operator 30 manipulates catheter 22 to perform electroanatomical mapping of a chamber of heart 26. EP signals are acquired from the myocardial tissue by bringing electrodes 48 on basket assembly 40 into contact with the tissue within the heart, as further detailed below.

In the pictured example, for purposes of position tracking, basket assembly 40 incorporates a pair of magnetic sensors 50A and 50B, seen in inset 45, at the proximal and distal ends of basket assembly 40. Alternatively, catheter 22 may comprise other sorts of magnetic sensors, at these or other locations. Alternatively or additionally, the catheter may comprise other sorts of position sensors, such as impedance-based or ultrasonic position sensors, as are known in the art.

Basket assembly 40 comprises multiple expandable spines 55, which are mechanically flexible. Multiple electrodes 48 are fixed to each spine, for a total of, for example, 120 electrodes. Electrodes 48 are configured to touch the tissue within heart 26 for the purpose of sensing EP signals, i.e., intracardiac electrogram signals in the pictured example. Magnetic sensors 50A and 50B and electrodes 48 are connected by wires (not shown) running through catheter 22 to processing circuits in a console 24.

Alternatively, system 20 may comprise other types of catheters, with other sorts of electrode arrays, such as an inflatable balloon catheter with electrodes 48 on its outer surface, or a catheter having one or more flexible arms or having a curved “lasso” at its distal end.

System 20 comprises a position-tracking sub-system 43 in console 24 for finding the position and orientation of basket assembly 40, and thereby identifying the locations of electrodes 48. Patient 28 is placed in a magnetic field generated by a pad containing magnetic field generator coils 42, which are driven by position-tracking sub-system 43. The magnetic fields generated by coils 42 give rise to electrical signals in sensors 50A and 50B, which are indicative of the position and orientation of the sensors. The signals from sensors 50A and 50B are transmitted back to position-tracking sub-system 43, which converts the signals to corresponding digital inputs to a processor 41. Processor 41 uses these inputs to compute the position and orientation of basket assembly 40 and thus to find the respective location coordinates of each of electrodes 48.

Alternatively or additionally, as noted above, system 20 may use other methods of position sensing to find the locations of electrodes 48. For example, processor 41 may map the locations of electrodes 48 by measuring impedances between electrodes 48 and body-surface electrodes 49, which are placed on the chest of patient 28 and connected to console 24 by leads 39.

Processor 41 additionally receives EP signals from electrodes 48 on basket assembly 40 via front-end circuits 44. These circuits apply analog and/or digital filters and amplifiers to the signals under the control of the processor. In a typical clinical application, processor 41 uses the information contained in these EP signals together with the coordinates provided by magnetic sensors 50A and 50B in constructing an electroanatomical map of the chamber of heart 26 in which basket assembly 40 is located, such as a map showing the voltage levels or local activation time (LAT) of the EP signals as a function of location along the chamber walls. In the present embodiment, however, processor 41 renders a graphical icon 60 to a display 27, representing basket assembly 40. Icon 60 incorporates visual indications of the quality of contact of electrodes 48 at the respective locations of the electrodes on the basket assembly. Methods for computation of metrics that are indicative of the quality of contact and their incorporation in icon 60 are described below.

Processor 41 is typically programmed in software to carry out the functions described herein. The software may be downloaded to the computer in electronic form, over a network, for example, or it may, alternatively or additionally, be provided and/or stored on non-transitory tangible media, such as magnetic, optical, or electronic memory. In particular, processor 41 runs a dedicated algorithm that enables the processor to perform the disclosed steps of data acquisition, computation of quality of contact, and operator guidance, as described below.

As noted earlier, the example illustration shown in FIG. 1 is chosen purely for the sake of conceptual clarity. FIG. 1 shows only elements related to the disclosed techniques for the sake of simplicity and clarity. System 20 typically comprises additional modules and elements that are not directly related to the disclosed techniques, and thus are intentionally omitted from FIG. 1 and from the corresponding description.

### Assessing and Displaying Quality of Contact

In response to signals provided by electrodes 48 on basket assembly 40, processor 41 assesses the respective quality of contact of each of the electrodes with the tissue in heart 26. Any one of electrodes 48 may be in full or partial contact with the tissue of the heart at any given time. Alternatively, any one of the electrodes may be separated from the tissue by a fluid, such as blood in the heart chamber, and will then receive signals from the tissue only through the fluid. The quality of contact (full or partial contact, or contact via fluid) of any one of the catheter electrodes with the tissue can be assessed based on the signals provided by the catheter. Based on these signals, processor 41 measures the quality of contact over a period during which basket assembly 40 moves within a chamber of the heart. The processor computes a metric for each electrode that is indicative of the quality of contact, for example as a function of the duration during which the electrode was in contact with the tissue over this period of time. In some embodiments, the metric for any given electrode corresponds to the number of valid signals acquired by the electrode during the period in question or to the fraction of the period during which there was good quality of contact between the electrode and the tissue.

The term “quality of contact,” as used in the specification and claims, is defined as a quantitative indicator of the degree of stable electrical contact between any one of the catheter electrodes and the tissue. The “quality of contact” may be expressed directly, for example in terms of a measured electrical impedance, or indirectly, for example in terms of contact force or pressure, or based on the amplitude of the EP signals that are acquired by electrodes 48. Methods for assessing the quality of contact between multiple electrodes on a catheter and tissue in the heart are described in detail in U.S. Patent Application Publication 2020/0367829, which is assigned to the assignee of the present patent application and whose disclosure is incorporated herein by reference with a copy in the Appendix.

Additionally or alternatively, the quality of contact may be expressed in terms of the quality of signal acquisition through the electrodes, for example based on the number of signals acquired by each electrode 48 that are found to meet certain filtering criteria, such as the criteria described in the above-mentioned U.S. patent application Ser. No. 16/995,036. In one embodiment using this sort of quality metric, processor 41 acquires a signal from each electrode 48 in the heart chamber within a certain time window of interest during each heartbeat period. (The start time of the heartbeat period, known as a “reference annotation,” is typically derived from the ECG signals received from body surface electrodes 49, and the window is defined relative to this reference annotation.) If the acquired signal and the electrode location during acquisition meet the filtering criteria, processor 41 counts the signal as valid and increments a count of the number of valid signals for this electrode. Otherwise, the signal is considered invalid and discarded. The contact quality metric for each electrode is based on the respective count.

By way of example, but not limitation, the filtering criteria used in counting the valid signals can include the following:
Proximity of electrodes to the wall of the heart chamber, for example based on the electrode location coordinates measure by the magnetic tracking system relative to the surface of the wall that has been reconstructed by a fast anatomical mapping (FAM) algorithm. Only the signals acquired at locations within a threshold distance of the wall are considered valid.
Catheter stability during acquisition. Processor 41 senses the extent and rate of motion of basket assembly 40 during acquisition of EP signals. When the basket assembly moves by more than a certain maximal distance during acquisition of an EP sample or set of samples, the processor will reject the signals as invalid.
Voltage too low. Processor 41 filters the EP signals by voltage level and will count as valid only the signals whose voltage was above a certain minimum.
   Only signals meeting all of the above criteria are counted as valid. The thresholds (for wall proximity, stability, and voltage, inter alia) can be fixed, or they can be set by an operator of system 20.

In other embodiments, processor 41 measures the impedance between electrodes 48 and body surface electrodes 49. The magnitude of the impedance provides an indication of a quality of contact. Typically, a higher value of impedance between one of the electrodes and the body surface electrodes indicates a higher quality of contact between that catheter electrode and the tissue, whereas low impedance indicates that the electrode is immersed in the blood within the heart. Processor 41 may use the values of impedance in computing the metrics indicating the quality contact between each of the catheter electrodes and the tissue.

Alternatively or additionally, the impedance between pairs of electrodes 48 on basket 40 may be used as a measure of quality of contact. Tissue contact may be assessed by comparing impedance values across a set of electrodes to premeasured impedance values, including values measured for electrodes known to be in sufficient contact with tissue and other values for electrodes known to be in contact only with blood.

Further additionally or alternatively, machine learning techniques may be used in assessing the quality of contact between electrodes 48 and myocardial tissue, for example as described in U.S. Pat. No. 9,168,004, whose disclosure is incorporated herein by reference with a copy in the Appendix.

In some embodiments, the probe under evaluation may comprise force or pressure sensors (not shown in the figures). The measure of force or pressure provides an indication of a quality of contact, such that a higher value of force or pressure indicates a higher quality of contact between a corresponding electrode and the tissue, and vice versa.

In some embodiments, the EP signals acquired from electrodes 48 are used to assess the quality of contact between the electrodes and the tissue. Processor 41 distinguishes between local signals acquired when an electrode is in contact with the tissue and far-field signals acquired when the electrode is not in contact with the tissue, and finds the quality of contact based on the relation between the local and far-field signals. For example, the maximum amplitude (voltage) of the EP signal associated with any given electrode is indicative of the quality of contact between the electrode and the tissue, such that a higher value of the maximum amplitude of the EP signal indicates a higher quality of contact between that catheter electrode and the tissue. Processor 41 may use the amplitude of the EP signal in computing the metric indicating the quality of contact between each of the catheter electrodes and the tissue.

Alternatively or additionally, processor 41 may apply other methods for measuring the quality of contact between electrodes and tissue 48, such as the methods that are further described in the above-mentioned U.S. Patent Application Publication 2020/0367829 or other methods that are known in the art.

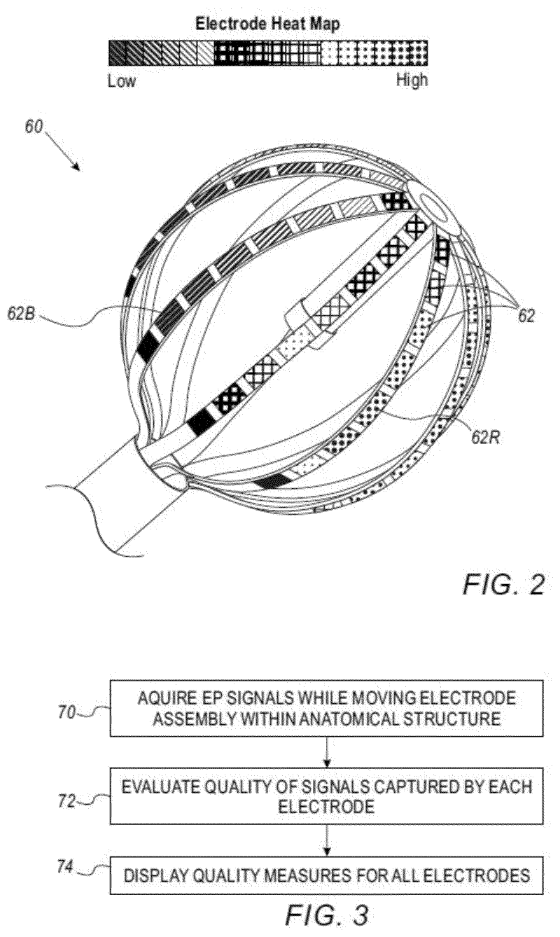

Reference is now made to FIGS. 2 and 3, which schematically illustrate a method for assessing and visualizing electrode performance, in accordance with an embodiment of the present invention. FIG. 2 is a schematic illustration of graphical icon 60 showing the quality of contact of electrodes on a catheter used in acquiring EP signals, while FIG. 3 is a flow chart showing the method for computation and display of contact quality metrics. The method is described here with specific reference to catheter 22 as shown in FIG. 1; but it may alternative be applied, mutatis mutandis, to catheters of other types.

Processor 41 typically renders icon 60 to display 27 and superimposes markings 62 on icon 60 corresponding to the respective locations of electrodes 48 on spines 55. Markings 62 are color-coded to indicate the respective contact quality metrics of the corresponding electrodes, for example using a “heat” scale (represented by different hatch styles in FIG. 2), with blue indicating the least contact and red indicating the most. Thus, in the pictured example, a location 62B made relatively poor contact with the myocardial tissue, while another location 62R made good contact.

The color-coding of markings 62 shows by implication which of the spines or which parts of the spines made frequent contact with the myocardial tissue and which did not. The designer of catheter 22 can then change the shapes of the spines or the distribution of the electrodes thereon in order to optimize the design. The contact metrics may be different when the catheter is used in different chambers of the art; and the designer may accordingly develop different baskets and electrode layouts for different applications. By the same token, an operator of system 20 may use the color-coding on icon 60 to improve his or her mapping technique in order to capture EP data more effectively.

As the first step in creating and coloring icon 60, processor 41 collects data with respect to electrodes 48 while basket 40 moves within an anatomical structure, such as a chamber of the heart, at an acquisition step 70, as shown in FIG. 3. In the present example, the acquired data comprises EP signals sensed by the electrodes, although other sorts of data, such as impedance or pressure measurements, may be acquired alternatively or additionally. Processor 41 measures the quality of contact made by each electrode with the tissue and computes a corresponding metric, at a quality evaluation step 72.

Based on this evaluation, the processor outputs an indication of the contact quality metric for each electrode, at a quality display step 74. For example, electrode positions 62 on icon 60 may be colored according to the quality metrics as illustrated in FIG. 2. Alternatively or additionally, other sorts of graphical and/or numerical outputs may be used, as will be apparent to those skilled in the art after reading the present description.

It will be appreciated that the embodiments described above are cited by way of example, and that the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art.

1. A system for electrophysiological measurement, comprising:
a probe having a distal end configured for insertion into a body cavity of a living subject and comprising an array of electrodes that are disposed along the distal end and are configured to contact tissue at multiple locations within the body cavity; and
a processor configured to:
acquire a plurality of signals from the electrodes over a period of time during which the probe moves within the body cavity,
compute, in response to the plurality of signals, a plurality of metrics, each metric being (i) associated with a respective electrode of the array of electrodes and (ii) indicative of a quality of contact between the respective electrode and the tissue, and
output an indication of each metric.

2. The system according to claim 1, wherein the distal end of the probe comprises a basket assembly on which the electrodes are arrayed, each metric is indicative of a contact between a part of the basket assembly and the tissue, and each part of the basket assembly associated with each respective metric is different from one another.

3. The system according to claim 1, wherein the distal end of the probe comprises a balloon on which the electrodes are arrayed, each metric is indicative of a contact between a part of the balloon and the tissue, and each part of the balloon associated with each respective metric is different from one another.

4. The system according to claim 1, wherein:
each electrode is disposed at a respective location on the distal end,
the processor is configured to render to a display a graphical icon representing the distal end and to incorporate, in the graphical icon, the indication of each metric at the respective location, and
each indication comprises a color-coded marking, the color-coded marking comprising a color that is based on the computed metric.

5. The system according to claim 1, wherein each metric is indicative of a number of valid signals acquired by the associated respective electrode from the tissue over the period of time.

6. The system according to claim 5, wherein the processor is configured to apply one or more filtering criteria to the signals in order to classify as valid a respective first set of the signals acquired from each of the electrodes while classifying as invalid a respective second set of the signals acquired by each of the electrodes.

7. The system according to claim 6, wherein the processor is configured to:
determine a proximity of each electrode to the tissue, and
classify the respective first set of the signals as valid based on the proximities being within a threshold distance of the tissue.

8. The system according to claim 6, wherein the processor is configured to classify the respective second set of the signals as invalid based on the probe moving more than a maximal distance during acquisition of the plurality of signals.

9. The system according to claim 6, wherein the processor is configured to:
filter the plurality of signals by voltage level, and
classify the respective first set of the signals as valid based on the voltage levels being above a predetermined minimum voltage level.

10. The system according to claim 1, wherein each metric is indicative of a respective duration during which the respective electrode was in contact with the tissue in the body cavity over the period of time.

11. A method comprising:
acquiring a plurality of signals from an array of electrodes, which are disposed along a distal end of a probe and are configured to contact tissue at multiple locations within a body cavity, within the body cavity over a period of time during which the probe moves within the body cavity;
computing, in response the plurality of signals, a plurality of metrics, each metric being (i) associated with a respective electrode of the array of electrodes and (ii) indicative of a quality of contact between the respective electrode and the tissue; and
outputting an indication of each metric.

12. The method according to claim 11, wherein the distal end of the probe comprises a basket assembly on which the electrodes are arrayed, each metric is indicative of a contact between a part of the basket assembly and the tissue, and each part of the basket assembly associated with each respective metric is different from one another.

13. The method according to claim 11, wherein the distal end of the probe comprises a balloon on which the electrodes are arrayed, each metric is indicative of a contact between a part of the balloon and the tissue, and each part of the balloon associated with each respective metric is different from one another.

14. The method accordingly to claim 11, wherein outputting the indication comprises:
rendering to a display a graphical icon representing the distal end, and
incorporating, in the graphical icon, the indication each metric at respective locations of the electrodes on the distal end, each incorporated indication comprising a color-coded marking, the color-coded marking comprising a color that is based on the computed metric.

15. The method according to claim 11, wherein each metric is indicative of a number of valid signals acquired by the associated respective electrode from the tissue over the period of time.

16. The method according to claim 15, wherein computing the metrics comprises applying one or more filtering criteria to the signals in order to classify as valid a respective first set of the signals acquired from each of the electrodes while classifying as invalid a respective second set of the signals acquired by each of the electrodes.

17. The method according to claim 16, wherein computing the metrics comprises:
determining a proximity of each electrode to the tissue, and
classifying the respective first set of the signals as valid based on the proximities being within a threshold distance of the tissue.

18. The method according to claim 16, wherein computing the metrics comprises:
classifying the respective second set of the signals as invalid based on the probe moving more than a maximal distance during acquisition of the plurality of signals.

19. The method according to claim 16, wherein computing the metrics comprises:
filtering the plurality of signals by voltage level, and
classifying the respective first set of the signals as valid based on the voltage levels being above a predetermined minimum voltage level.

20. The method according to claim 11, wherein each metric is indicative of a respective duration during which the respective electrode was in contact with the tissue in the body cavity over the period of time.
